(19) United States
(12) Patent Application Publication
(10) Pub. No.: US 2023/0125363 A1
(43) Pub. Date: Apr. 27, 2023

(54) Polyamideimide Precursor, Polyamideimide Prepared Therefrom, and Polyamideimide Film Including the Same

(71) Applicants: SK Innovation Co., Ltd., Seoul (KR); SK ie technology Co., Ltd., Seoul (KR)

(72) Inventors: Jong Chan Kim, Daejeon (KR); Jin Hyung Park, Daejeon (KR); Seung Min Jeon, Daejeon (KR); Hyo Shin Kwak, Daejeon (KR); So Young Lee, Daejeon (KR)

(21) Appl. No.: 17/972,745

(22) Filed: Oct. 25, 2022

(30) Foreign Application Priority Data
Oct. 26, 2021 (KR) 10-2021-0143655

(51) Int. Cl. C08G 73/14

(52) U.S. Cl. CPC C08G 73/14

(57) ABSTRACT

Provided are a polyamideimide precursor, a polyamideimide prepared therefrom, and a polyamideimide film including the same. Specifically, the polyamideimide film according to an exemplary embodiment includes a polyamideimide derived from a combination of specific compositions to implement a high modulus and excellent optical properties.

### CROSS-REFERENCE TO RELATED APPLICATION

This application claims priority to Korean Patent Application No. 10-2021-0143655, filed Oct. 26, 2021, the disclosure of which is hereby incorporated by reference in its entirety.

### BACKGROUND OF THE INVENTION

### Field of the Invention

The following disclosure relates to a polyamideimide precursor, a polyamideimide prepared therefrom, and a polyamideimide film including the same.

More particularly, the following disclosure relates to a polyamideimide film which includes a polyamideimide derived from a combination of specific compositions to implement a high modulus and excellent optical properties.

### Description of Related Art

In recent years, as light weight, slimness, and flexibility of a display device have been regarded as important, a study for replacing a glass substrate, a cover glass, and the like which have been widely used in conventional displays with a polyimide is being actively conducted. In order to apply the polyimide to a next-generation display device, the polyimide should have not only excellent optical properties but also improved mechanical properties, and thus, the performance required for a polyimide-based polymer for a display device is gradually advanced.

To this end, a study for improving mechanical properties by combining a monomer having strong straightness and rigidity with or introducing an amide group into a transparent polyimide-based resin (colorless polyimide, CPI) is in progress. However, the optical properties and the mechanical properties of a polyimide-based resin are in a trade-off relationship, and the attempt has a limitation of deteriorated optical properties even in the case in which the mechanical properties of the polyimide-based resin are improved. In addition, since the solution handleability of the polyimide-based resin is deteriorated, a process difficulty is increased or it becomes impossible to obtain resin.

Thus, the development of a polyimide-based film which may further broaden the scope of application by implementing improved mechanical properties, in particular, a high modulus, while having colorless and transparent performance, is demanded.

### SUMMARY OF THE INVENTION

An embodiment is directed to providing a polyamideimide film which may improve both excellent mechanical properties and optical properties.

Another embodiment is directed to providing a polyamideimide precursor prepared with a combination of specific monomers, which may provide a polyamideimide film having the above physical properties.

Another embodiment is directed to providing a high-strength, colorless and transparent polyamideimide film by using the polyamideimide precursor.

Still another embodiment is directed to providing a multilayer structure including the polyamideimide film and a display device including the same.

In one general aspect, a polyamideimide precursor includes: a structural unit derived from an aromatic diamine, a structural unit derived from a dianhydride, and a structural unit derived from an aromatic diacid dichloride, wherein the aromatic diamine includes a compound represented by the following Chemical Formula 1:

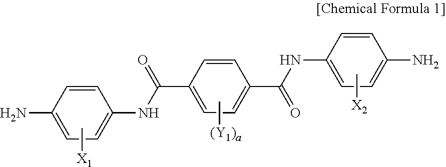

wherein X₁, X₂, and Y₁ are independently of one another fluoro(C1-C7)alkyl, perfluoro(C1-C7)alkyl, or a fluoro group; and a is an integer of 0 to 4.

The compound represented by Chemical Formula 1 according to an exemplary embodiment may be represented by the following Chemical Formula 2 or Chemical Formula 3:

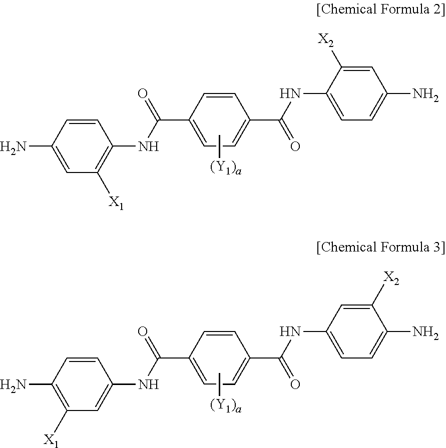

wherein X₁, X₂, and Y₁ are independently of one another perfluoro(C1-C7)alkyl or a fluoro group; and a is an integer of 0 to 4.

The compound represented by Chemical Formula 1 according to an exemplary embodiment may be selected from the following structures:

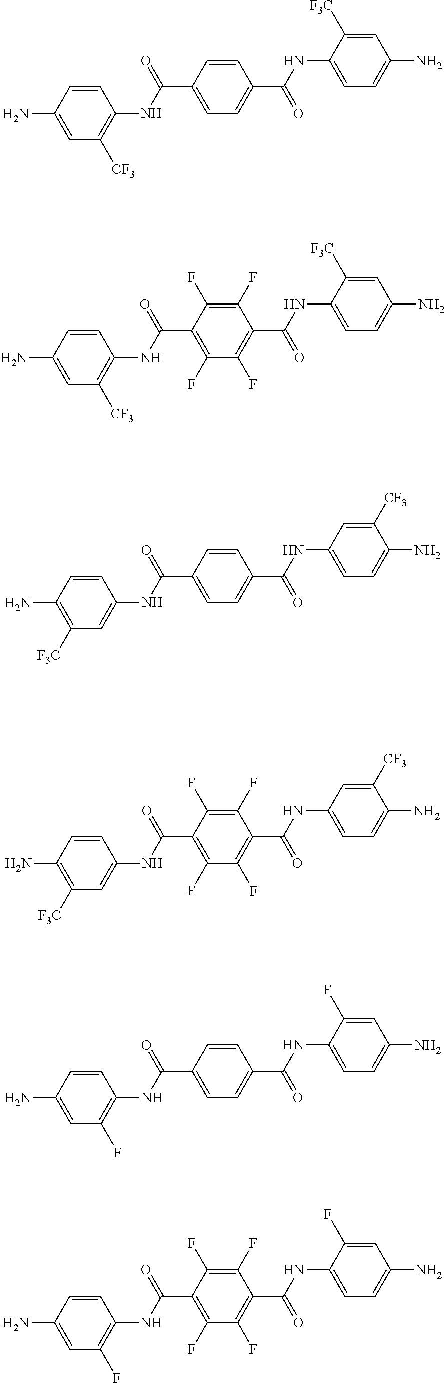 [SMILES: Nc1ccc(NC(=O)c2ccc(C(=O)Nc3ccc(N)cc3C(F)(F)F)cc2)c(C(F)(F)F)c1 ; Nc1ccc(NC(=O)c2c(F)c(F)c(C(=O)Nc3ccc(N)cc3C(F)(F)F)c(F)c2F)c(C(F)(F)F)c1 ; Nc1ccc(NC(=O)c2ccc(C(=O)Nc3ccc(N)c(C(F)(F)F)c3)cc2)cc1C(F)(F)F ; Nc1ccc(NC(=O)c2c(F)c(F)c(C(=O)Nc3ccc(N)c(C(F)(F)F)c3)c(F)c2F)cc1C(F)(F)F ; Nc1ccc(NC(=O)c2ccc(C(=O)Nc3ccc(N)cc3F)cc2)c(F)c1 ; Nc1ccc(NC(=O)c2c(F)c(F)c(C(=O)Nc3ccc(N)cc3F)c(F)c2F)c(F)c1]

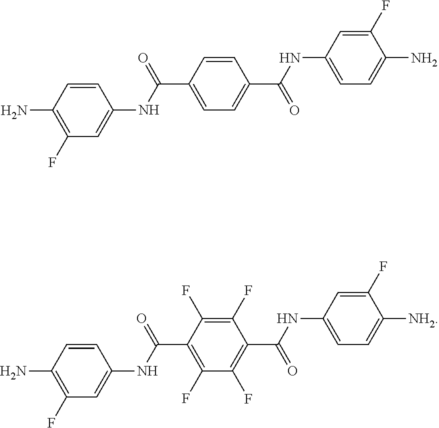 [SMILES: Nc1ccc(NC(=O)c2ccc(C(=O)Nc3ccc(N)c(F)c3)cc2)cc1F ; Nc1ccc(NC(=O)c2c(F)c(F)c(C(=O)Nc3ccc(N)c(F)c3)c(F)c2F)cc1F]

The aromatic diamine according to an exemplary embodiment may further include a second aromatic diamine which is different from the compound represented by Chemical Formula 1.

The second aromatic diamine according to an exemplary embodiment may contain an aromatic ring substituted with a trifluoroalkyl group.

The second aromatic diamine according to an exemplary embodiment may be 2,2′-bis(trifluoromethyl)-benzidine.

The structural unit derived from the compound represented by Chemical Formula 1 according to an exemplary embodiment may be included at 5 to 30 mol % based on the total moles of the structural unit derived from an aromatic diamine.

The dianhydride according to an exemplary embodiment may include an aromatic dianhydride and an alicyclic dianhydride.

The aromatic dianhydride according to an exemplary embodiment may include 4, 4′-(hexafluoroisopropylidene)-diphthalic anhydride, and the alicyclic dianhydride may include 1,2,3,4-cyclobutanetetracarboxylic dianhydride.

The aromatic diacid dichloride according to an exemplary embodiment may include terephthaloyl dichloride, isophthaloyl dichloride, 1,1′-biphenyl-4,4′-dicarbonyl dichloride, 1,4-naphthalene dicarboxylic dichloride, 2,6-naphthalene dicarboxylic dichloride, 1,5-naphthalene dicarboxylic dichloride, or a combination thereof.

The structural unit derived from an aromatic diacid dichloride according to an exemplary embodiment may be included at 50 mol to 90 mol with respect to 100 mol of the structural unit derived from an aromatic diamine.

In another general aspect, a polyamideimide prepared from the polyamideimide precursor is provided.

In another general aspect, a composition for forming a polyamideimide film includes: the polyamideimide precursor, a polyamideimide prepared from the polyamideimide precursor, or a combination thereof.

In another general aspect, a polyamideimide film formed of the composition for forming a polyamideimide film is provided.

The polyamideimide film according to an exemplary embodiment may have a thickness of 20 to 500 μm, a modulus in accordance with ASTM D882 of 7 GPa or more, a haze in accordance with ASTM D1003 of 3.0 or less, and a total light transmittance measured in 400 to 700 nm in accordance with ASTM D1003 of 85% or more.

In another general aspect, a window cover film includes the polyamideimide film.

In still another general aspect, a display device includes the window cover film.

Other features and aspects will be apparent from the following detailed description, and the claims.

### DESCRIPTION OF THE INVENTION

In the present invention, unless otherwise defined, all technical terms and scientific terms have the same meanings as those commonly understood by a person skilled in the art to which the present invention pertains. The terms used herein are only for effectively describing a certain specific example, and are not intended to limit the present invention.

The singular form used in the present specification may be intended to also include a plural form, unless otherwise indicated in the context.

The term “comprise” in the present specification is an open-ended description having a meaning equivalent to the term such as “is/are provided”, “contain”, “have”, or “is/are characterized”, and does not exclude elements, materials, or processes which are not further listed.

The term “combination thereof” in the present specification may refer to mixing or copolymerization of constituents.

The term “polymer” in the present specification includes an oligomer, and includes a homopolymer and a copolymer. The copolymer may include an alternating copolymer, a block copolymer, a random copolymer, a branch copolymer, a crosslinked copolymer, or all of them.

The term “polyamic acid” refers to a polymer including a structural unit having an amic acid moiety, the term “polyimide” refers to a polymer including a structural unit having an imide moiety, the term “polyamide” refers to a polymer including a structural unit having an amide moiety, the term “polyamideimide” refers to a polymer including a structural unit having an imide moiety and an amide moiety, and the term “poly(amide-amic acid)” refers to a polymer including a structural unit having an amic acid moiety and an amide moiety, in the present specification. The term “polyamideimide precursor solution” in the present specification may have an equivalent meaning to a “polyamic acid amide solution”, and refer to a solution including polyamideimide and/or polyamic acid amide. In addition, the “polyimide” may be used in the meaning including polyimide or polyamideimide, and the “polyamic acid” may be used in the meaning including polyamic acid or polyamic acid amide.

The term “alkyl” in the present specification is an organic radical derived from an aliphatic hydrocarbon by removal of one hydrogen, and may include both a straight chain and branched chain forms. The alkyl may have 1 to 10 carbon atoms, specifically 1 to 7 carbon atoms, or specifically 1 to 5 carbon atoms. The alkyl includes, as an example, methyl, ethyl, propyl, isopropyl, butyl, isobutyl, t-butyl, pentyl, hexyl, ethylhexyl, and the like, but is not limited thereto.

In order to apply a polyimide film to a display device, the intrinsic yellow index properties should be improved, the colorless and transparent performance should be secured, and the mechanical properties should be improved in the polyimide film. However, when a compound having a rigid structure or an amide group is introduced for improving the mechanical properties of a colorless polyimide (CPI), the mechanical properties may be improved, but optical properties may be deteriorated. In addition, solution handleability is lowered to increase process difficulty and there may be limitation in obtaining a film using the resin. Thus, an attempt to obtain a new polyimide-based resin which may impart both excellent mechanical properties and optical properties to a polyimide film and has excellent handleability is being actively made.

The polyamideimide precursor according to an exemplary embodiment includes a structural unit derived from a combination of specific monomer compositions, thereby providing a polyamideimide film having both improved optical properties and improved mechanical properties.

Specifically, since the polyamideimide precursor according to an exemplary embodiment is prepared from a combination of specific aromatic diamine, dianhydride, and aromatic diacid dichloride, it may provide a polyamideimide film showing a high light transmittance throughout the visible light region, having a low haze, and having excellent mechanical strength including a high modulus.

Specifically, the polyamideimide precursor according to an exemplary embodiment may include: a structural unit derived from an aromatic diamine, a structural unit derived from a dianhydride, and a structural unit derived from an aromatic diacid dichloride, wherein the aromatic diamine includes a compound represented by the following Chemical Formula 1:

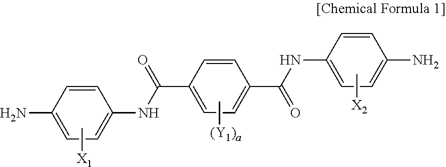

wherein X₁, X₂, and Y₁ are independently of one another fluoro(C1-C7)alkyl, perfluoro(C1-C7)alkyl, or a fluoro group; and a is an integer of 0 to 4.

The compound of Chemical Formula 1 includes a plurality of aromatic rings to improve the mechanical strength of a film. Simultaneously, a substituent including fluoro in the aromatic ring may be introduced to decrease a charge transfer complex (CTC) effect. In addition, a packing density between the chains or in the structure of polyamideimide is lowered, and also, even with a sufficient thickness thereof, a film having significantly improved optical properties may be provided. Though it is not bound to a specific theory, as an example, when a substituent including fluoro is not introduced as described above, CTC is greatly expressed to make optical properties vulnerable, and solution handleability is so poor that preparation of a film is impossible.

In addition, the aromatic diamine of Chemical Formula 1 may have a plurality of amide bonds to decrease n-conjugation, thereby more effectively decreasing the CTC effect.

The polyamideimide precursor according to an exemplary embodiment is prepared from a combination of a dianhydride and an aromatic diacid dichloride monomer with an aromatic diamine including a compound having a specific structure like Chemical Formula 1, thereby providing a polyamideimide film capable of implementing both excellent mechanical strength and optical properties.

Specifically, the compound represented by Chemical Formula 1 may be represented by the following Chemical Formula 2 or 3:

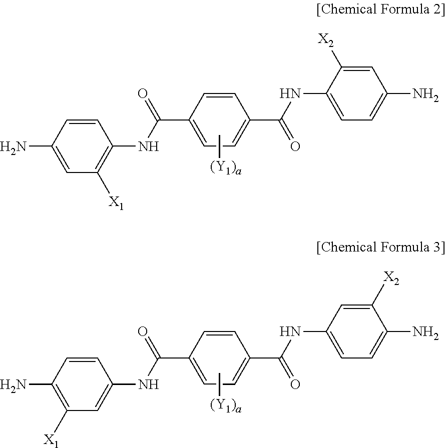

wherein X₁, X₂, and Y₁ are independently of one another perfluoro(C1-C7)alkyl or a fluoro group; and a is an integer of 0 to 4.

As an example, X₁ and X₂ may be identical to each other, and may be perfluoro(C1-C5)alkyl or a fluoro group.

As an example, Y₁ may be a fluoro group, and a may be 0 or 4.

More specifically, the compound represented by Chemical Formula 1 according to an exemplary embodiment may be selected from the following structures, but is not limited thereto:

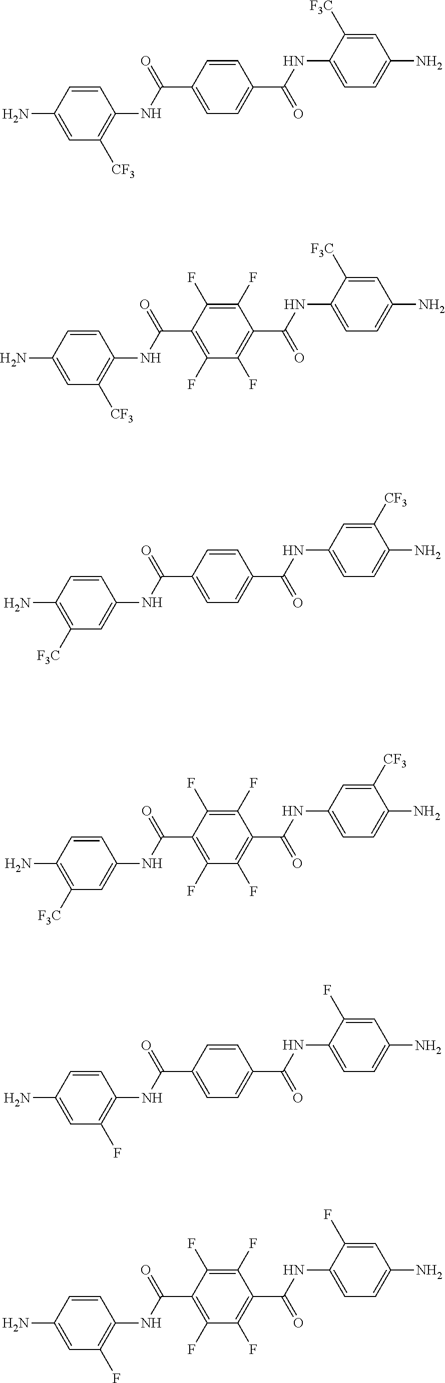 [SMILES: Nc1ccc(NC(=O)c2ccc(C(=O)Nc3ccc(N)cc3C(F)(F)F)cc2)c(C(F)(F)F)c1 ; Nc1ccc(NC(=O)c2c(F)c(F)c(C(=O)Nc3ccc(N)cc3C(F)(F)F)c(F)c2F)c(C(F)(F)F)c1 ; Nc1ccc(NC(=O)c2ccc(C(=O)Nc3ccc(N)c(C(F)(F)F)c3)cc2)cc1C(F)(F)F ; Nc1ccc(NC(=O)c2c(F)c(F)c(C(=O)Nc3ccc(N)c(C(F)(F)F)c3)c(F)c2F)cc1C(F)(F)F ; Nc1ccc(NC(=O)c2ccc(C(=O)Nc3ccc(N)cc3F)cc2)c(F)c1 ; Nc1ccc(NC(=O)c2c(F)c(F)c(C(=O)Nc3ccc(N)cc3F)c(F)c2F)c(F)c1]

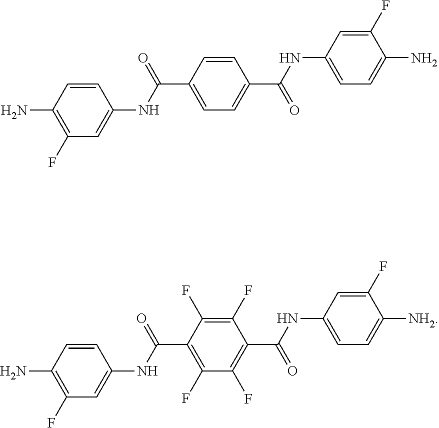 [SMILES: Nc1ccc(NC(=O)c2ccc(C(=O)Nc3ccc(N)c(F)c3)cc2)cc1F ; Nc1ccc(NC(=O)c2c(F)c(F)c(C(=O)Nc3ccc(N)c(F)c3)c(F)c2F)cc1F]

Specifically, the polyamideimide film according to an exemplary embodiment may maintain a high light transmittance and low haze properties well, and also, implement a synergistic effect of significantly improving a modulus.

In an exemplary embodiment, as the aromatic diamine, the compound represented by Chemical Formula 1 is used alone, or if necessary, aromatic diamines commonly used in the art are mixed therewith, so that two or more aromatic diamine compounds may be used.

As an example, in an exemplary embodiment, the aromatic diamine may further include a second aromatic diamine which is different from the compound represented by Chemical Formula 1, in addition to the compound represented by Chemical Formula 1. The second aromatic diamine may include a substituted or unsubstituted C6-C30 aromatic ring, in which the aromatic ring may be a single ring; a fused ring in which two or more aromatic rings are fused; or a non-fused ring in which two or more aromatic rings are connected by a single bond, a C1-5 alkylene group, or O or C(═O).

As an example, the second aromatic diamine may be an aromatic diamine compound to which a fluorine-based substituent is introduced, and the fluorine-based substituent may include fluoro(C1-C7)alkyl, perfluoro(C1-C7)alkyl, or a fluoro group. Specifically, the second aromatic diamine may include an aromatic ring substituted with one or two or more trifluoroalkyl groups, and the aromatic ring substituted with a trifluoroalkyl group may be unsubstituted or further substituted with a substituent other than the trifluoroalkyl group.

More specifically, the second aromatic diamine may be 2,2′-bis(trifluoromethyl)benzidine (TFMB). This may induce a charge transfer effect of fluorine substituents to impart better optical properties to the film.

As an example, when the aromatic diamine further includes an aromatic diamine different from the compound represented by Chemical Formula 1, the structural unit derived from the compound represented by Chemical Formula 1 may be included at 5 to 30 mol %, specifically 10 to 30 mol % based on the total moles of the structural unit derived from the entire aromatic diamines. When the range is satisfied, an effect of improving both mechanical properties and optical properties of the polyamideimide film may be better when other monomer constituent components are combined, which is thus preferred, but the present disclosure is not necessarily limited thereto.

The dianhydride according to an exemplary embodiment may include an aromatic dianhydride, an alicyclic dianhydride, or a combination thereof, and for example, the dianhydride may include an aromatic dianhydride and an alicyclic dianhydride.

The aromatic dianhydride refers to a dianhydride including at least one aromatic ring, in which the aromatic ring is as described above. The aromatic dianhydride may be, for example, one or two or more selected from 9,9-bis(3,4-dicarboxyphenyl)fluorene dianhydride (BPAF), 4,4′-(hexafluoroisopropylidene)diphthalic anhydride (6FDA), 3,3′,4,4′-biphenyltetracarboxylic dianhydride (BPDA), 4,4′-oxydiphthalic anhydride (ODPA), sulfonyldiphthalic anhydride (SO2DPA), (isopropylidenediphenoxy)bis(phthalic anhydride (6HDBA), 4-(2,5-dioxotetrahydrofuran-3-yl)-1,2,3,4-tetrahydronaphthalene-1,2-dicarboxylic anhydride (TDA), 1,2,4,5-Benzenetetracarboxylic anhydride (PMDA), and benzophenone tetracarboxylic dianhydride (BTDA), but is not limited thereto.

Specifically, the aromatic dianhydride may be a fluorine-based aromatic dianhydride compound to which a fluorine-based substituent is introduced, and the fluorine-based substituent may include fluoro(C1-C7)alkyl, perfluoro(C1-C7)alkyl, or a fluoro group. Specifically, the aromatic dianhydride may include an aromatic ring substituted with one or two or more trifluoroalkyl groups, and the aromatic ring substituted with a trifluoroalkyl group may be unsubstituted or further substituted with a substituent other than the trifluoroalkyl group. For example, the aromatic dianhydride may be 6FDA. Since the fluorine-based aromatic dianhydride described above is used, the optical properties of the polyamideimide film may be improved, and mechanical strength, in particular, modulus may be improved more effectively.

The alicyclic dianhydride refers to a dianhydride including at least one aliphatic ring, and the aliphatic ring may be a single ring; a fused ring in which two or more aliphatic rings are fused; or a non-fused ring in which two or more aliphatic rings are connected by a single bond, a substituted or unsubstituted (C1-C5) alkylene group, or O or C(═O). For example, the alicyclic dianhydride may be any one or two or more selected from the group consisting of 1,2,3,4-cyclobutanetetracarboxylic dianhydride (CBDA), 5-(2,5-Dioxotetrahydrofuryl)-3-methyl-3-cyclohexene-1,2-dicarboxylic anhydride (DOCDA), bicyclo[2.2.2]oct-7-ene-2,3,5,6-tetracarboxylic dianhydride (BTA), bicyclooctene-2,3,5,6-tetracarboxylic dianhydride (BODA), 1,2,3,4-cyclopentanetetracarboxylic dianhydride (CPDA), 1,2,4,5-cyclohexanetetracarboxylic dianhydride (CHDA), 1,2,4-tricarboxy-3-methylcarboxycyclopentane dianhydride (TMDA), 1,2,3,4-tetracarboxycyclopentane dianhydride (TCDA), and derivatives thereof, but is not limited thereto. Specifically, the alicyclic dianhydride may be CBDA.

More specifically, the dianhydride compound according to an exemplary embodiment may be a combination of an aromatic dianhydride and an alicyclic dianhydride, and for example, may be a combination of a fluorine-based aromatic dianhydride and an alicyclic dianhydride, and when a combination of 6FDA and CBDA is used, the effect of improving mechanical properties and optical properties of the polyamideimide film may be better when the aromatic diamine and the aromatic diacid dichloride as described above are combined.

The aromatic diacid dichloride according to an exemplary embodiment is reacted with the diamine compound described above to form an amide structure in a polymer chain, whereby the mechanical properties including a modulus may be better in a range of not deteriorating the optical properties of the film.

The aromatic diacid dichloride may be one or two or more selected from the group consisting of isophthaloyl dichloride (IPC), terephthaloyl dichloride (TPC), [1,1′-biphenyl]-4,4′-dicarbonyl dichloride (BPC), 1,4-naphthalene dicarboxylic dichloride (NPC), 2,6-naphthalene dicarboxylic dichloride (NTC), 1,5-naphthalene dicarboxylic dichloride (NEC), 4,4′-oxybis(benzoylchloride) (DEDC), and derivatives thereof, and for example, may be TPC, but is not limited thereto.

In an exemplary embodiment, the content of the aromatic diacid dichloride is not particularly limited, but for example, may be included at 50 mol or less, or 50 mol or more, with respect to 100 mol of the aromatic diamine. Even in the case in which the aromatic diacid dichloride is included at 50 mol or more with respect to 100 mol of the aromatic diamine, since the diamine of Chemical Formula 1 is included, the optical properties maintained excellent and the mechanical properties may be further improved.

The structural unit derived from an aromatic diacid dichloride according to an exemplary embodiment may be used at 50 mol to 90 mol, specifically 60 mol to 90 mol, or 60 mol to 80 mol with respect to 100 mol of the structural unit derived from an aromatic diamine, but is not necessarily limited thereto.

When the aromatic diacid dichloride is combined with other monomers in the range described above, the optical properties and mechanical strength of the polyamideimide film may be better. Specifically, long light transmittance and low haze properties are implemented, and a synergistic effect of more significantly improving a modulus may be implemented.

In addition, the polyamideimide precursor according to an exemplary embodiment may be prepared by adding the aromatic diamine, dianhydride, and the aromatic diacid dichloride together, or may be prepared by reacting the aromatic diamine and the aromatic diacid dichloride to prepare a polyamide oligomer having an amine end and then reacting the polyamide oligomer with an additional aromatic diamine and a dianhydride, but is not necessarily limited thereto. When the polyamide oligomer having an amine end is prepared and then is reacted with an additional aromatic diamine and a dianhydride, a block polyamideimide may be prepared, and the mechanical properties of the film may be further improved.

In addition, an exemplary embodiment provides a polyamideimide prepared from the polyamideimide precursor.

In addition, an exemplary embodiment provides a composition for forming a polyamideimide film including the polyamideimide precursor and/or the polyamideimide.

Specifically, the composition for forming a polyamideimide film may include the polyamideimide precursor according to an exemplary embodiment, the polyamideimide prepared from the polyamideimide precursor, or a mixture thereof; and an organic solvent.

The composition for forming a polyamideimide film according to an exemplary embodiment includes the polyamideimide precursor and/or the polyamideimide as described above, thereby providing a polyamideimide film having significantly improved mechanical properties. In particular, the composition according to an exemplary embodiment may provide a polyamideimide film having a significantly improved modulus value while maintaining colorless and transparent properties.

The organic solvent included in the composition according to an exemplary embodiment may be one or two or more selected from ketones such as γ-butyrolactone, 1,3-dimethyl-2-imidazolidinone, methyl ethyl ketone, cyclohexanone, cyclopentanone, and 4-hydroxy-4-methyl-2-pentanone; aromatic hydrocarbons such as toluene, xylene, and tetramethylbenzene; glycolethers such as ethylene glycol monoethyl ether, ethylene glycol monomethy ether, ethylene glycol monobutyl ether, diethylene glycol monoethylether, diethylene glycol monomethylether, diethylene glycol monobutyl ether, propylene glycol monomethyl ether, propylene glycol monoethyl ether, dipropylene glycol diethyl ether, and triethylene glycol monoethyl ether; acetates such as ethyl acetate, butyl acetate, ethylene glycol monoethyl ether acetate, ethylene glycol monobutyl ether acetate, diethylene glycol monoethyl ether acetate, and dipropylene glycol monomethyl ether acetate; alcohols such as methanol, ethanol, propanol, ethylene glycol, propylene glycol, and carbitol; amides such as N,N-dimethylpropionamide (DMPA), N,N-diethylpropionamide(DEPA), N,N-dimethylacetamide (DMAc), N,N-diethylacetamide (DEAc), N,N-dimethylformamide (DMF), N,N-diethylformamide (DEF), N-methylpyrrolidone (NMP), N-ethylpyrrolidone (NEP), and N,N-dimethylmethoxyacetamide; and the like, but is not limited thereto.

Specifically, the organic solvent may be one or a mixture of two or more selected from the amides described above. As an example, it may be N,N-dimethylacetamide (DMAc), N,N-diethylacetamide (DEAc), N-methylpyrrolidone (NMP), M-ethylpyrrolidone (NEP), N,N-dimethylpropionamide (DMPA), N,N-diethylpropionamide (DEPA), or a combination thereof.

The solid content of the composition for forming a polyamideimide film according to an exemplary embodiment may be adjusted to an amount to allow the composition to have an appropriate viscosity, considering the applicability in a subsequent process of forming a film. As an example, the composition for forming a polyamideimide film according to an exemplary embodiment may have a solid content of 5 to 40 wt %, 10 to 30 wt %, or 10 to 20 wt %, based on the total weight of the composition. The solid content refers to the polyamideimide precursor and/or the polyamideimide.

Specifically, the composition for forming a polyamideimide film according to an exemplary embodiment may have a viscosity of 5,000 to 100,000 cps or 15,000 to 50,000 cps when the solid content is satisfied. When the viscosity range described above is satisfied, the efficiency of defoaming in the processing of the polyamideimide film may be better to provide a process advantage. Thus, a more uniform surface may be implemented. Here, the viscosity refers to a value measured using a Brookfield DV2TRV viscometer spindle No. 52, after placing a sample at room temperature (25° C.), and subjecting the sample to a stabilization operation when a torque value is at 80%.

In addition, an exemplary embodiment provides a polyamideimide film prepared using the composition for forming a polyamideimide film.

The polyamideimide film according to an exemplary embodiment includes a structural unit derived from an aromatic diamine including the compound represented by Chemical Formula 1, a dianhydride, and an aromatic diacid dichloride, thereby having excellent transparency, a high modulus, and excellent mechanical strength.

The polyamideimide film according to an exemplary embodiment may have a thickness of 10 to 500 μm, for example, 10 to 300 μm, for example, 20 to 100 μm, and for example, 30 to 100 μm.

In addition, the polyamideimide film according to an exemplary embodiment may have a modulus in accordance with ASTM D882 of 6 GPa or more, 7 GPa or more, or 7.2 GPa or more.

In addition, the polyamideimide film according to an exemplary embodiment satisfies the modulus value, and also, may have a haze in accordance with ASTM D1003 of 5.0 or less, 3.0 or less, or 2.0 or less and a total light transmittance in accordance with ASTM D1003 of 80% or more, 85% or more, or 87% or more.

That is, the polyamideimide film according to an exemplary embodiment maintains colorless and transparent properties even in a thickness range of 20 to 100 um, and also implements excellent mechanical strength.

The polyamideimide film according to an exemplary embodiment includes a structural unit derived from a combination of specific monomer compositions, thereby implementing excellent optical properties and mechanical properties as described above. Specifically, an exemplary embodiment includes structural units derived from an aromatic diamine including the compound represented by Chemical Formula 1, a dianhydride, and an aromatic diacid dichloride, thereby providing a polyamideimide film having excellent optical properties, mechanical strength, and flexibility. Therefore, the polyamideimide film according to an exemplary embodiment may be applied to various fields such as a substrate for a device, a cover substrate for a display, an optical film, an integrated circuit (IC) package, a deposition film, a multilayer flexible printed circuit (FPC), a tape, a touch panel, and a protective film for an optical disc.

Hereinafter, a method of preparing a polyimide film according to an exemplary embodiment will be described in detail.

The polyamideimide film according to an exemplary embodiment may be prepared by chemical curing or thermal curing, and a stretching step may be further included.

The chemical curing may be performed by chemical imidization of the polyamideimide precursor. Specifically, it may include: chemically imidizing the polyamideimide precursor solution according to an exemplary embodiment to prepare a polyamideimide resin; and applying a resin composition including the polyamideimide resin (composition for forming a polyamideimide film) to form a film.

The chemical imidization may be performed by further including any one or two or more selected from an imidization catalyst and a dehydrating agent. The imidization catalyst may be any one or two or more selected from pyridine, isoquinoline, β-quinoline, and the like. In addition, the dehydrating agent may be any one or two or more selected from an acetic anhydride, a phthalic anhydride, a maleic anhydride, and the like, but is not necessarily limited thereto. Here, after the imidization of the polyamideimide precursor is performed, the product is precipitated in a solvent and purified to obtain a solid content (polyamideimide powder), which is dissolved in an organic solvent to adjust the solid content, thereby obtaining a composition for forming a film.

The film forming step may be applying the composition for forming a polyamideimide film on a substrate, and then forming a film by drying by a heat treatment. The substrate may be, for example, glass, stainless, or a film, and the like, and the application may be performed by a die coater, an air knife, a reverse roll, spray, blade, casting, gravure, spin coating, and the like, but is not limited thereto.

The heat treatment may be performed, for example, step by step. For example, it may be performed by a stepwise heat treatment of primary drying at 70° C. to 160° C. for 1 minute to 2 hours and secondary drying at 150° C. to 350° C. for 1 minute to 2 hours. However, the heat treatment is not necessarily limited to the temperature and time conditions, and for example, the primary drying may be performed at 80° C. to 150° C., 70° C. to 110° C., 130° C. to 150° C., 90° C., 120° C., or 140° C. for 10 minutes to 90 minutes, 10 minutes to 60 minutes, 20 minutes to 50 minutes, or 30 minutes, and the secondary drying may be performed at 200° C. to 300° C., 220° C. to 300° C., or 250° C. to 300° C. for 10 minutes to 90 minutes, 30 minutes to 90 minutes, or 40 minutes to 80 minutes. Here, the stepwise heat treatment may be performed preferably by heating in a range of 1 to 20° C/min at each step moving. In addition, the heat treatment may be performed in a separate vacuum oven, an oven filled with inert gas, or the like, but is not necessarily limited thereto.

In addition, the thermal curing according to an exemplary embodiment may be performed by thermal imidization of the polyamideimide precursor. Specifically, it may be performed by applying a composition for forming a polyamideimide film including a polyamideimide precursor or a mixture of a polyamideimide precursor and polyamideimide on a substrate, and performing thermal curing.

The thermal curing may be performed at 100 to 450° C., 120 to 450° C., or 150 to 450° C. More specifically, the thermal curing may be performed at 80 to 100° C. for 1 minute to 2 hours, at higher than 100° C. and 200° C. or lower for 1 minute to 20 hours, or at higher than 200° C. and 450° C. or lower for 1 minute to 2 hours, and stepwise thermal curing may be performed under two or more temperature conditions selected therefrom. In addition, the thermal curing may be performed in a separate vacuum oven, an oven filled with inert gas, or the like, but is not necessarily limited thereto. In addition, before the thermal curing step, a drying step may be further performed, if necessary. The drying step may be performed at a temperature of 50° C. to 150° C., 50° C. to 130° C., 60° C. to 100° C., or about 80° C., but is not necessarily limited thereto.

In addition, the composition for forming a polyamideimide film may be further mixed with one or two or more additives selected from a flame retardant, an adhesive strength improver, inorganic particles, an anti-oxidant, a UV blocking agent, a plasticizer, and the like to prepare the polyamideimide film.

In addition, the polyamideimide film according to an exemplary embodiment may be provided as a multilayer structure which is a form including two or more layers.

Specifically, the multilayer structure may further include a functional coating layer on at least one other surface of the polyamideimide film or the substrate. A non-limiting example of the functional coating layer may include a hard coating layer, an antistatic layer, an anti-fingerprint layer, an antifouling layer, an anti-scratch layer, a low refractive index layer, an anti-reflection layer, an impact absorbing layer, and the like, and at least one or two or more functional coating layers may be provided.

In addition, in an exemplary embodiment, various forms of molded articles may be manufactured using the polyamideimide film. An example of the molded article may be applied to a printed wiring board, a flexible circuit board, and the like including a film, a protective film, or an insulating film, but is not limited thereto. Specifically, it may be applied to a protective film which may replace a cover glass, and has a wide application range in various industrial fields including a display.

More specifically, it may be used as a window cover film such as a flexible display.

The polyamideimide film according to an exemplary embodiment includes a structural unit derived from an aromatic diamine including the compound represented by Chemical Formula 1, a dianhydride, and an aromatic diacid dichloride, thereby implementing excellent optical properties such as high transparency and excellent modulus. Accordingly, the polyamideimide film according to an exemplary embodiment may be used as a window cover film of a flexible display panel and the like. The window cover including the polyamideimide film according to an exemplary embodiment has better optical properties to have excellent visibility, has a high modulus and excellent mechanical strength, and thus, may be used as an alternative material for tempered glass.

Hereinafter, examples will be provided for specifically describing the present invention, but the present invention is not limited to the examples below.

The physical properties of the examples were measured as follows:

(1) Weight Average Molecular Weight

A weight average molecular weight was measured by dissolving a film in a DMAc eluent containing 0.05 M LiCl. Waters GPC system, Waters 1515 isocratic HPLC Pump, and Waters 2414 Refractive Index detector were used as GPC, Olexis, Polypore and a mixed D column were connected as a column, and polymethylmethacrylate (PMMA STD) was used as a standard material. Analysis was performed at 35° C. at a flow rate of 1 mL/min.

(2) Viscosity

A Brookfield DV2TRV viscometer spindle No. 52 was used after placing a sample at room temperature (25° C.) and subjecting the sample to a stabilization operation for 2 minutes when a torque value is at 80%, and the results were measured. A viscosity unit was cps.

(3) Total Light Transmittance

A light transmittance was measured using a spectrophotometer (Nippon Denshoku, COH-400) in an entire wavelength region of 400 to 700 nm in accordance with the ASTM D1003 specification, based on the films having a thickness of 50 pm prepared in the examples and the comparative examples.

(4) Haze

A haze was measured, using a spectrophotometer (Nippon Denshoku, COH-400) in accordance with the ASTM D1003 specification, based on the films having a thickness of 50 μm prepared in the examples and the comparative examples.

(5) Modulus

A modulus was measured by using UTM 3365 available from Instron under the condition of pulling the polyamideimide films having a thickness of 50 μm, a length of 50 mm, and a width of 10 mm prepared in the examples and the comparative examples at 25° C. at 50 mm/min, in accordance with ASTM D882. The unit of the modulus was GPa.

### PREPARATION EXAMPLE 1

### Preparation of Diamine Compound 1

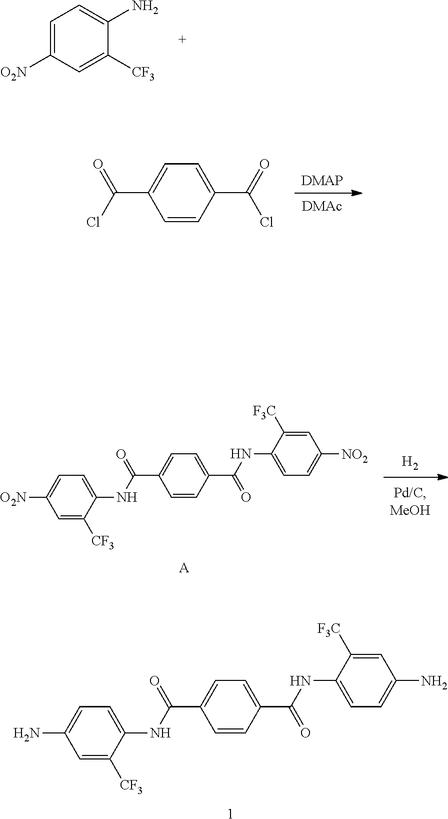

Preparation of Compound A

30.0 g (145.5 mmol) of 4-nitro-2-(trifluoromethyl)aniline, 17.4 g (72.8 mmol) of terephthaloyl dichloride (TPC), 35.6 g (291.1 mmol) of 4-(dimethylamino)pyridine, and 300 mL of N,N-dimethylacetamide (DMAc) were added to a round bottom flask under a nitrogen atmosphere, heated to 80° C., and stirred for 12 hours. The solution was cooled to room temperature, and then 200 mL of water was added to a filtrate after filtration with a Celite pad and strong stirring was performed to precipitate a solid, which was filtered again to obtain 34.6 g of a solid. 200 mL of methanol (MeOH) was added, stirring was performed at room temperature (25° C.) for 2 hours, and filtration was performed to obtain a reaction product, which was dried to obtain 32.8 g (yield: 83%) of Compound A which was an ivory solid.

¹H-NMR (DMSO-d6): 10.65 ppm (2H, s), 8.58-8.60 ppm (2H, q), 8.53-8.54 ppm (2H, d), 8.13 ppm (4H, s), 7.97-7.99 ppm (2H, d)

Preparation of Diamine Compound 1

32.8 g of the obtained Compound A, 80 mL of methanol (MeOH), and 3 g of Pd/C (10 wt % Pd basis) were added to a 1 L autoclave, and stirring was performed. The inside of the autoclave was replaced with hydrogen, and a hydrogenation reaction was performed at 40° C. for 24 hours by adding hydrogen with pressure until the internal pressure was 10 bar. After the reaction was completed, a transparent supernatant was poured, 400 mL of N,N-dimethylformamide (DMF) was added thereto, and stirring was performed to perform dissolution. The solution was passed through a Celite pad to remove Pd/C, concentrated, heated to 120° C. in a reaction solution state, and cooled to room temperature (25° C.) to crystallize the solid. The produced solid was filtered, and dried under vacuum to obtain 18.4 g (yield: 66%) of Diamine Compound 1 as a final product.

¹H-NMR (DMSO-d6): 9.86 ppm (2H, s), 8.01 ppm (4H, s), 7.09-7.11 ppm (2H, s), 6.93 ppm (2H, s), 6.81-6.83 ppm (2H, s), 5.61 ppm (4H, s)

### EXAMPLE 1

2,2′-bis(trifluoromethyl)-benzidine (TFMB) was added to N,N-dimethylacetamide (DMAc) in a reactor under a nitrogen atmosphere, stirring was sufficiently performed, terephthaloyl dichloride (TPC) was added thereto, and stirring was performed for room temperature (25° C.) for 6 hours to perform dissolution and reaction. Thereafter, a reaction product obtained by precipitation and filtration using excessive water was dried under vacuum at 90° C. for 6 hours or more to obtain a polyamide oligomer having a number average molecular weight of 1,700 g/mol.

The polyamide oligomer, additional TFMB, and Diamine Compound 1 obtained in Preparation Example 1 were added to the reactor under a nitrogen atmosphere, and the amount of the aromatic diamine used was the amount such that the mole ratio of TFMB:Diamine Compound 1 is 70:30. Thereafter, cyclobutanetetracarboxylic dianhydride (CBDA) and 4,4′-hexafluoroisopropylidene diphthalic anhydride (6FDA) were sequentially added, so that the total amount of each monomer used was TFMB:Diamine Compound 1:TPC:6FDA:CBDA=70:30:55:15:30 as a mole ratio. The mixed solution was stirred at 40° C. for 12 hours, dissolved, and reacted to prepare a polyamideimide precursor solution.

Subsequently, each of pyridine and acetic anhydride was added sequentially to the polyamideimide precursor solution at 2.5-fold relative the total content of dianhydride, and stirring was performed at 60° C. for 12 hours to prepare a composition including a polyamideimide resin (composition for forming a polyamideimide film). The viscosity of the prepared composition was 24,300 cps, and the content of the final solid content was 10 wt %.

The polyamideimide resin solution was cast on a glass substrate using an applicator. Thereafter, first drying was performed in a drying oven at 90° C. for 30 minutes, a heat treatment was performed in a curing oven at 300° C. for 30 minutes under a N2 condition, cooling to room temperature was performed, and a film formed on the glass substrate was separated from the substrate to obtain a polyamideimide film having a thickness of 50 μm. The physical properties of the thus-obtained polyamideimide film are shown in the following Table 1.

### EXAMPLES 2 AND 3

A polyamideimide film was obtained in the same manner as in Example 1, except that the amount of monomer added was changed as shown in the following Table 1. The physical properties of the prepared sample were measured, and are shown in the following Table 1.

### COMPARATIVE EXAMPLE 1

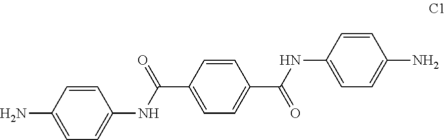 [SMILES: Nc1ccc(NC(=O)c2ccc(C(=O)Nc3ccc(N)cc3)cc2)cc1]

A polyamideimide film was obtained in the same manner as in Example 1, except that Compound Cl was used instead of Diamine Compound 1. The physical properties of the prepared sample were measured, and are shown in the following Table 1.

### COMPARATIVE EXAMPLE 2

A polyamideimide film was obtained in the same manner as in Example 1, except that Diamine Compound 1 was not used, and the amount of monomer added was changed as shown in the following Table 1. The physical properties of the prepared sample were measured, and are shown in the following Table 1.

### COMPARATIVE EXAMPLE 3

A polyamideimide film was obtained in the same manner as in Example 1, except that terephthaloyl dichloride (TPC) was not used, and the amount of monomer added was changed as shown in the following Table 1. The physical properties of the prepared sample were measured, and are shown in the following Table 1.

TABLE 1

| | Composition (mole ratio) | | | | | | | | | |
|---|---|---|---|---|---|---|---|---|---|---|
| | | Diamine | | | | | Thickness | Modulus | Transmittance | |
| | TFMB | Compound 1 | C1 | TPC | 6FDA | CBDA | (μm) | (GPa) | (%) | Haze |
| Example 1 | 70 | 30 | | 55 | 15 | 30 | 50 | 7.82 | 88.50 | 1.2 |
| Example 2 | 80 | 20 | | 55 | 15 | 30 | 50 | 7.55 | 88.61 | 0.75 |
| Example 3 | 90 | 10 | | 55 | 15 | 30 | 50 | 7.34 | 89.01 | 0.56 |
| Comparative | 70 | | 30 | 55 | 15 | 30 | Unable to prepare film | | | |
| Example 1 | | | | | | | | | | |
| Comparative | 100 | | | 55 | 15 | 30 | 50 | 6.1 | 90.5 | 0.34 |
| Example 2 | | | | | | | | | | |
| Comparative | 90 | 10 | | | 80 | 20 | 50 | 5.7 | 89.24 | 0.82 |
| Example 3 | | | | | | | | | | |

As shown in Table 1, in Examples 1 to 3 using a monomer combination of an aromatic diamine including the compound represented by Chemical Formula 1, a dianhydride, and an aromatic diacid dichloride, it was confirmed that a film having a sufficient thickness was able to be formed. However, in Comparative Example 1, a film was not able to be prepared with a high packing density.

In addition, all of the polyamideimide films of Examples 1 to 3 had excellent optical properties and mechanical strength. Specifically, it was confirmed that the films had a significantly improved modulus value while maintaining optical properties equivalent to those of the polyamideimide films of Comparative Examples 2 and 3.

The polyamideimide film prepared from the polyamideimide precursor according to an exemplary embodiment may implement both excellent mechanical properties and optical properties.

Specifically, the polyamideimide precursor is prepared by a combination of structures having both a unit capable of improving mechanical properties and a unit capable of decreasing a charge transfer complex (CTC) effect, thereby more effectively improving mechanical strength without a decrease in a transparency of a polyamideimide film.

In addition, the polyamideimide precursor according to an exemplary embodiment may have excellent solution handleability to improve preparation processability, and allows preparation of a film having a sufficient thickness.

The polyamideimide precursor according to an exemplary embodiment may provide a polyamideimide film which is colorless and transparent and has excellent mechanical strength, and may also have excellent preparation processability and be applied to various industrial fields including displays.

Hereinabove, although the present invention has been described by specific exemplary embodiments, they have been provided only for assisting in the entire understanding of the present invention. Therefore, the present invention is not limited to the exemplary embodiments. Various modifications and changes may be made by those skilled in the art to which the present invention pertains from this description.

Therefore, the spirit of the present invention should not be limited to the above-described exemplary embodiments, and the following claims as well as all modified equally or equivalently to the claims are intended to fall within the scope and spirit of the invention.

1. A polyamideimide precursor comprising: a structural unit derived from an aromatic diamine, a structural unit derived from a dianhydride, and a structural unit derived from an aromatic diacid dichloride,
wherein the aromatic diamine comprises a compound represented by the following Chemical Formula 1: 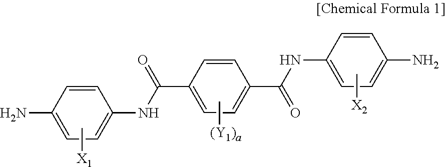
wherein X₁, X₂, and Y₁ are independently of one another fluoro(C1-C7)alkyl, perfluoro(C1-C7)alkyl, or a fluoro group;
and a is an integer of 0 to 4.

2. The polyamideimide precursor of claim 1, wherein the compound represented by Chemical Formula 1 is represented by the following Chemical Formula 2 or Chemical Formula 3: 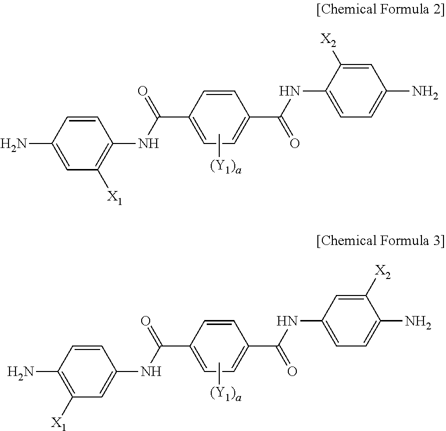
wherein X₁, X₂, and Y₁ are independently of one another perfluoro(C1-C7)alkyl or a fluoro group; and a is an integer of 0 to 4.

3. The polyamideimide precursor of claim 1, wherein the compound represented by Chemical Formula 1 is selected from the following structures:

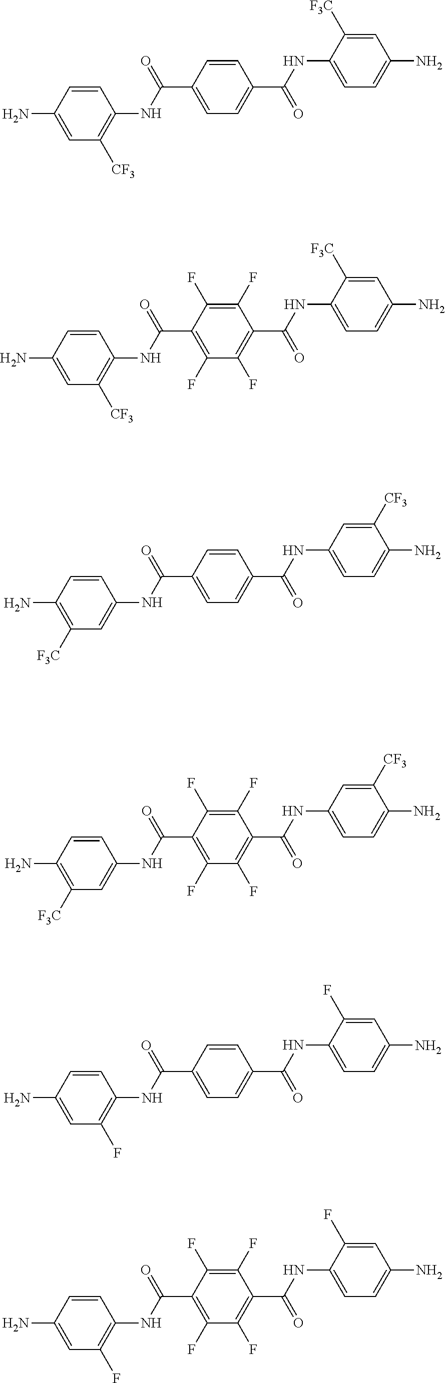 [SMILES: Nc1ccc(NC(=O)c2ccc(C(=O)Nc3ccc(N)cc3C(F)(F)F)cc2)c(C(F)(F)F)c1 ; Nc1ccc(NC(=O)c2c(F)c(F)c(C(=O)Nc3ccc(N)cc3C(F)(F)F)c(F)c2F)c(C(F)(F)F)c1 ; Nc1ccc(NC(=O)c2ccc(C(=O)Nc3ccc(N)c(C(F)(F)F)c3)cc2)cc1C(F)(F)F ; Nc1ccc(NC(=O)c2c(F)c(F)c(C(=O)Nc3ccc(N)c(C(F)(F)F)c3)c(F)c2F)cc1C(F)(F)F ; Nc1ccc(NC(=O)c2ccc(C(=O)Nc3ccc(N)cc3F)cc2)c(F)c1 ; Nc1ccc(NC(=O)c2c(F)c(F)c(C(=O)Nc3ccc(N)cc3F)c(F)c2F)c(F)c1]

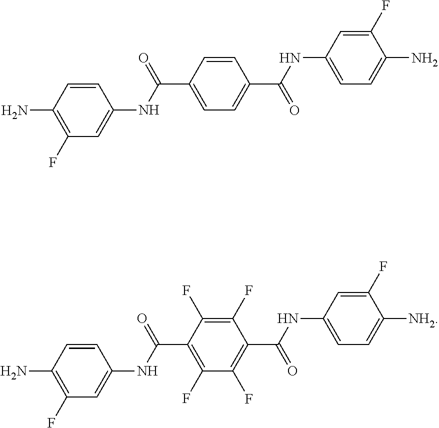 [SMILES: Nc1ccc(NC(=O)c2ccc(C(=O)Nc3ccc(N)c(F)c3)cc2)cc1F ; Nc1ccc(NC(=O)c2c(F)c(F)c(C(=O)Nc3ccc(N)c(F)c3)c(F)c2F)cc1F]

4. The polyamideimide precursor of claim 1, wherein the aromatic diamine further comprises a second aromatic diamine which is different from the compound represented by Chemical Formula 1.

5. The polyamideimide precursor of claim 4, wherein the second aromatic diamine contains an aromatic ring substituted with a trifluoroalkyl group.

6. The polyamideimide precursor of claim 4, wherein the second aromatic diamine is 2,2′-bis(trifluoromethyl)-benzidine.

7. The polyamideimide precursor of claim 1, wherein the structural unit derived from the compound represented by Chemical Formula 1 is comprised at 5 to 30 mol % based on total moles of the structural unit derived from an aromatic diamine.

8. The polyamideimide precursor of claim 1, wherein the dianhydride comprises an aromatic dianhydride and an alicyclic dianhydride.

9. The polyamideimide precursor of claim 8, wherein the aromatic dianhydride comprises 4,4′-(hexafluoroisopropylidene)-diphthalic anhydride, and the alicyclic dianhydride comprises 1,2,3,4-cyclobutanetetracarboxylic dianhydride.

10. The polyamideimide precursor of claim 1, wherein the aromatic diacid dichloride comprises terephthaloyl dichloride, isophthaloyl dichloride, 1,1′-biphenyl-4,4′-dicarbonyl dichloride, 1,4-naphthalene dicarboxylic dichloride, 2,6-naphthalene dicarboxylic dichloride, 1,5-naphthalene dicarboxylic dichloride, or a combination thereof.

11. The polyamideimide precursor of claim 1, wherein the structural unit derived from an aromatic diacid dichloride is comprised at 50 mol to 90 mol with respect to 100 mol of the structural unit derived from an aromatic diamine.

12. The polyamideimide prepared from the polyamideimide precursor of claim 1.

13. A composition for forming a polyamideimide film comprising:
the polyamideimide precursor of claim 1, a polyamideimide prepared from the polyamideimide precursor, or a combination thereof.

14. A polyamideimide film formed of the composition for forming a polyamideimide film of claim 13.

15. The polyamideimide film of claim 14, wherein the polyamideimide film has a thickness of 20 to 500 μm, a modulus in accordance with ASTM D882 of 7 GPa or more, a haze in accordance with ASTM D1003 of 3.0 or less, and a total light transmittance measured in 400 to 700 nm in accordance with ASTM D1003 of 85% or more.

16. A window cover film comprising the polyamideimide film of claim 14.

17. A display device comprising the window cover film of claim 16.
